(19) United States
(12) Patent Application Publication
(10) Pub. No.: US 2010/0191273 A1
(43) Pub. Date: Jul. 29, 2010

(54) EMBOLIC PROTECTION DEVICE WITH NO DELIVERY CATHETER OR RETRIEVAL CATHETER AND METHODS OF USING THE SAME

(75) Inventor: Karl KEATING, Oughterard (IE)

Correspondence Address: Sughrue Mion-ABBOTT LABS, 2100 Pennsylvania Avenue, N.W., Washington, DC, 20037, US

(73) Assignee: SALVIAC LIMITED, Dublin (IE)

(21) Appl. No.: 12/358,282

(22) Filed: Jan. 23, 2009

(51) Int. Cl. A61F 2/01

(52) U.S. Cl. 606/200

(57) ABSTRACT

A device and method for filtering emboli from blood has been formed. The device includes an expandable filter capable of being delivered and retrieved from a treatment site in a low-profile, collapsed position without the use of a delivery catheter or a retrieval catheter.

### FIELD OF THE INVENTION

The present invention relates generally to medical devices, and in particular, to an embolic protection device that does not require the use of a delivery catheter or a retrieval catheter and methods of using the same.

### BACKGROUND OF THE INVENTION

The term “STROKE” is used to describe a medical event whereby blood supply to the brain or specific areas of the brain is restricted or blocked to the extent that the supply is inadequate to provide the required flow of oxygenated blood to maintain function. The brain will be impaired either temporarily or permanently, with the patient experiencing a loss of function such as sight, speech or control of limbs. There are two distinct types of stroke, hemorrhagic and embolic. Embolic stroke may be caused by embolic material that may become dislodged after stenting.

Medical literature describes artery disease as a significant source of embolic material. Typically, an atherosclerotic plaque builds up in the arteries. The nature of the plaque varies considerably, but in a significant number of cases pieces of the plaque can break away and flow distally and, for example, block blood flow to specific areas of the brain and cause neurological impairment, plaque can also break free and flow into the lungs or heart and cause other adverse events. Treatment of the disease in the carotid artery is classically by way of surgical carotid endarterectomy whereby, the carotid artery is cut and the plaque is physically removed from the vessel. The procedure has broad acceptance with neurological complication rates quoted as being low, somewhere in the order of 5% although claims vary widely on this.

Not all patients are candidates for surgery. A number of reasons may exist such that the patients could not tolerate surgical intervention. In these cases and an increasing number of candidates that are surgical candidates are being treated using transcatheter techniques. In this case, the evolving approach uses devices inserted in the femoral artery and manipulated to the site of the stenosis. A balloon angioplasty catheter is inflated to open the artery and an intravascular stent is sometimes deployed at the site of the stenosis. The action of these devices as with surgery can dislodge embolic material which will flow with the arterial blood and if large enough, eventually block a blood vessel and cause a stroke.

It is known to permanently implant a filter in human vasculature, such as the vena cava, to catch embolic material. It is also known to use a removable filter for this purpose. Such removable filters typically comprise umbrella type filters comprising a filter membrane supported on a collapsible frame on a guidewire for movement of the filter membrane between a collapsed position against the guidewire and a laterally extending position occluding a vessel. Examples of such filters are shown in U.S. Pat. No. 4,723,549, U.S. Pat. No. 5,053,008 and U.S. Pat. No. 5,108,419. Various deployment and/or collapsing arrangements are provided for the umbrella filter.

Improved filter devices such as those shown in U.S. Pat. No. 6,336,934, U.S. Pat. No. 6,551,342 and U.S. Patent Application Publication No. 2003/0065354, the entireties of which are hereby incorporated by reference, have been designed to overcome the shortcomings of the previous filters. For example, in one embodiment, the filter is freely disposed along the length to the guidewire, thereby allowing the guidewire to be moved independently of the filter assembly.

After the filter has crossed the stenosed region of the vessel, the filter is deployed within the vessel to capture any emboli that may be dislodged during subsequent medical procedure(s). However, one problem associated with current embolic protection filter devices is that they must cross the stenosed area(s) before they can be deployed. In some instances the stenosed area may have a restricted diameter such that the filter may drag or knock loose some of the plaque during crossing, thereby causing emboli to be released within the lumen. The emboli may cause complications such as stroke or possibly death.

In particular, these embolic protection devices typically require the use of a delivery catheter for the purpose of delivering the filter device to the treatment site (that is, a self-expanding filter device may be in a collapsed condition within the end of the catheter and is typically expanded by pushing or filling the filter out of the distal end of the catheter). Because of the inherent thickness of the delivery catheter, the increased profile of the embolic protection device during delivery may increase the risk of plaque being dislodged when the catheter crosses the stenosed area(s). In addition, retrieval of the filter after an interventional procedure, such as stenting, requires an additional step wherein a second catheter is employed to retrieve the filter and withdraw it from the vasculature (typically, the filter is collapsed by urging the proximal end of the filter into the retrieval catheter, i.e., the catheter provides the force for collapsing the filter). Advancing the retrieval catheter to the filter also carries a risk of stent snagging.

Therefore, there is a need for an improved embolic protection device, wherein the device has a low-profile that reduces the risk of emboli formation while crossing a stenosed portion of the lumen and reduces the risk of stent snagging. Particularly, there is the need for an improved embolic protection device that does not require the use of a catheter for any one of delivering, retrieving, expanding or collapsing an embolic protection filter.

### BRIEF SUMMARY

In accordance with an embodiment of the present invention, there is provided an embolic protection device comprising: (1) a first elongate member having a proximal end and a distal end; (2) a second elongate member having a lumen, a proximal end a distal end, the first elongate member slidably disposed within the lumen of the second elongate member; (3) a guiding tip having a proximal end and a distal end, the proximal end of the guiding tip coupled to the distal end of the second elongate member; and (4) an expandable filter disposed on the guiding tip, wherein the filter is movable between a low-profile, collapsed position and an expanded position by distally translating the first elongate member relative to the second elongate member.

In one embodiment, the embolic protection device further comprises a frame having a proximal end and a distal end, wherein the proximal end of the frame is coupled to the first elongate member and the distal end of the frame is coupled to the guiding tip.

In a further embodiment, the frame is movable between a low-profile, collapsed position and an expanded position, and the filter is supported by the frame when the frame is in its expanded position.

In a preferred embodiment, the frame is self-expanding.

In another embodiment, the embolic protection device further comprises a tether having a proximal end and a distal end, wherein the proximal end of the tether is coupled to the first elongate member and the distal end of the tether is coupled to the filter.

In a further embodiment, the tether is movable between a low-profile, collapsed position and an expanded position.

In a further embodiment, the tether is substantially received within the lumen of the second elongate member when the tether is in its low-profile, collapsed position.

In a preferred embodiment, the tether is self-collapsing.

In a preferred embodiment, the tether holds the filter, in tension, on the embolic protection device when the filter is in its low-profile, collapsed position.

In accordance with another embodiment of the present invention there is provided a method for filtering emboli from blood flowing within a vasculature at a delivery location comprising the steps of: (1) providing the embolic protection device according to the embodiment described above; (2) collapsing the filter by translating the first elongate member proximally relative to the second elongate member to move the filter into the low-profile, collapsed position; (3) advancing the embolic protection device to the delivery location within the vasculature; (4) deploying the filter at the delivery location by translating the first elongate member distally relative to the second elongate member to move the filter to the expanded position; (5) performing an interventional procedure at a position proximal to the delivery location; (6) collapsing the filter after the interventional procedure by translating the first elongate member proximally relative to the second elongate member to move the filter to the low-profile, collapsed position; and (7) withdrawing the embolic protection device from the vasculature.

In a further embodiment, the interventional procedure comprises advancing a stent over the second elongate member to the position proximal to the delivery location.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings.

FIG. 1 is a perspective view of an exemplary embodiment of an actuator according to the present invention.

FIG. 2 is a perspective view of an exemplary embodiment of a core shaft according to the present invention.

FIG. 3 is a perspective view of an exemplary embodiment of a guiding tip according to the present invention.

FIG. 4A is a plan view of an exemplary embodiment of a shaft assembly according to the present invention.

FIG. 4B is a partial perspective view of a exemplary embodiment of a shaft assembly according to the present invention.

FIG. 5A is a perspective view of an exemplary embodiment of a filter according to the present invention.

FIG. 5B is a cross-sectional view of two exemplary embodiments of a filter according to the present invention.

FIG. 5C is a partial perspective view of an exemplary embodiment of a filter according to the present invention.

FIG. 5(d) is a perspective view of an exemplary embodiment of a filter disposed on a shaft assembly according to the present invention.

FIG. 6A is a perspective view of an exemplary embodiment of a tether according to the present invention.

FIG. 6B is a perspective view of an exemplary embodiment of a tether attached to a filter and shaft assembly according to the present invention.

FIG. 7A is a perspective view of an exemplary embodiment of a spiral frame according to the present invention.

FIG. 7B is a perspective view of an exemplary embodiment of a spiral frame attached to shaft assembly and supporting a filter according to the present invention.

FIG. 8 is a perspective view of an exemplary embodiment of an embolic protection device according to the present invention.

FIG. 9 is a partial perspective view of an exemplary embodiment of an embolic protection device according to the present invention.

FIG. 10A is a plan view of an exemplary embodiment of an embolic protection device of the present invention in use.

FIG. 10B is a plan view of an exemplary embodiment of an embolic protection device of the present invention in use.

FIG. 10C is a plan view of an exemplary embodiment of an embolic protection device of the present invention in use.

It should be noted that the figures are not drawn to scale and that elements of similar structures or functions are generally represented by like reference numerals for illustrative purposes throughout the figures. It also should be noted that the figures are only intended to facilitate the description of embodiments of the present invention.

### DETAILED DESCRIPTION

It is to be understood that both the foregoing general description and the following detailed description are exemplary and are intended to provide further explanation of the invention claimed.

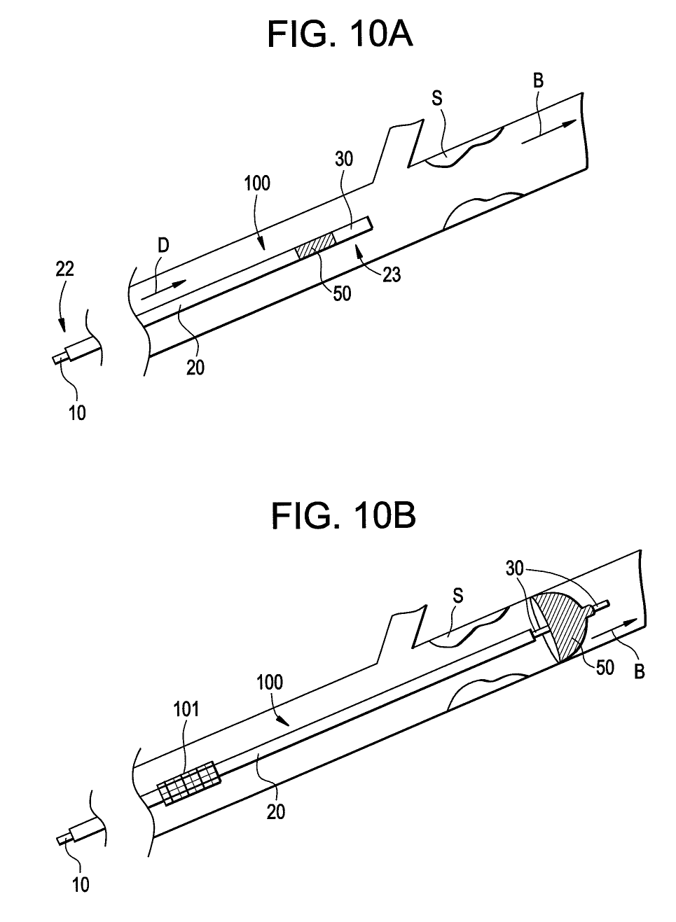

Referring initially to FIG. 8, in accordance with an embodiment of the present invention, there is shown an embolic protection device comprising an actuator 10; a core shaft 20; a guiding tip 30; and an expandable filter 50. The elongated body of the actuator 10 is slidably received within the elongated, hollow body of the core shaft 20, and the guiding tip 30 is coupled to, and extends from, the distal end 23 of the core shaft 20. The combination of the actuator 10, core shaft 20 and guiding tip 30 are collectively referred to herein as a shaft assembly.

By translating the actuator 10 relative to the core shaft 20, as described in further detail below, the expandable filter 50 is movable between a low-profile, collapsed position, not shown, and the expanded position illustrated in FIG. 8. The filter 50 may comprise support elements. For example, one or more tether 60 may be employed as a first support element to hold the expandable filter 50, in tension, on the shaft assembly when the filter 50 is in its low-profile, collapsed position. In a preferred embodiment, a self-collapsing tether is coupled between the filter 50 and the actuator 10. In addition, one or more frame 70 may be employed as a second support element to aid in the expansion of the filter 50 from its low-profile, collapsed position and/or to support the body of the filter 50 in its expanded position. In a preferred embodiment, a self-expanding frame 70 is coupled between the actuator 10 and the guiding tip 30 and the body of the filter 50 disposed around the frame 70.

The elements of the illustrated embodiment of an embolic protection device according to the present invention that are described generally above are explained in further detail below.

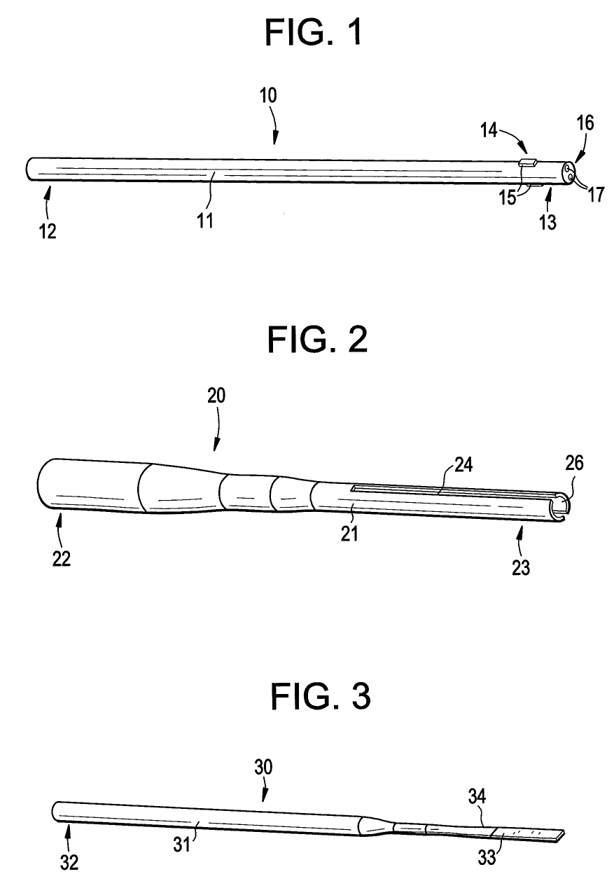

Referring now to FIG. 1, there is shown an exemplary embodiment of an actuator 10 in accordance with the present invention. The actuator 10 includes a first elongate member 11 having a proximal end 12 and a distal end 13. In the illustrated embodiment, the first elongate member 11 has an elongated, rod-like shape. However, the shape of the first elongate member 11 is not particularly limited. The first elongate member 11 may be made from a metal, polymer or composite material, preferably a biocompatible material. The first elongate member 11 is generally flexible, but is relatively stiff in both axial and radial directions to allow proper placement within the vasculature, similar to a typical catheter or guidewire.

The actuator 10 may be composed of multiple components. For example, in FIG. 1 the actuator 10 includes a tether attachment feature 14 associated with the distal end 13 of the first elongate member 11. In the illustrated embodiment, the tether attachment feature 14 is shown as two rectangular protrusions 15. However, the constitution of the tether attachment feature 16 is not particularly limited. Particular uses and functions of the tether attachment feature 14 are described further below.

The actuator 10 may also include a frame attachment feature 16 associated with the distal end of the first elongate member 11. In the illustrated embodiment, the frame attachment feature 16 is shown as two circular bores 17. However, the constitution of the frame attachment feature 16 is not particularly limited. Particular uses and functions of the frame attachment feature 17 are described further below.

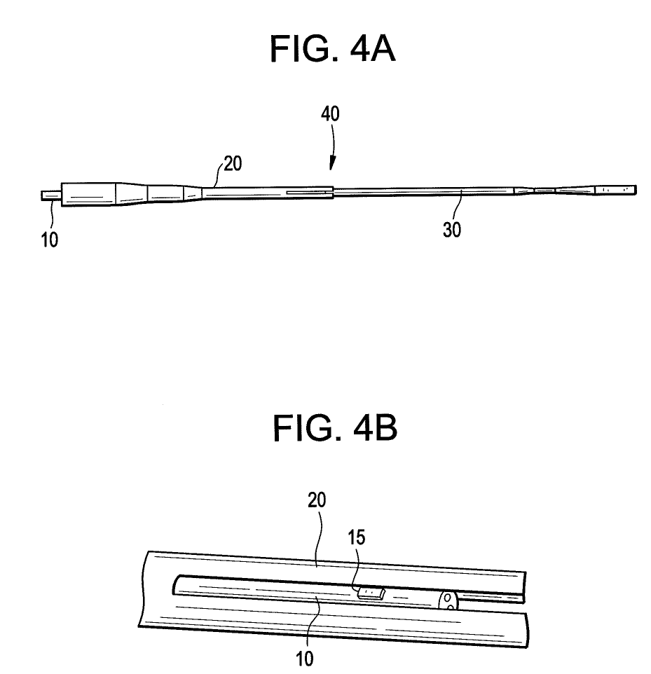

Referring now to FIG. 2, there is shown an exemplary embodiment of a core shaft 20 in accordance with the present invention. The core shaft 20 includes a second elongate member 21 having a proximal end 22 and a distal end 23. The second elongate member 21 includes a lumen 26 that is adapted to slidably receive the first elongate member 11. The second elongate member 21 is generally flexible, but is relatively stiff in both axial and radial directions to allow proper placement within the vasculature, similar to a typical catheter or guidewire.

The general shape of the core shaft 20 is not particularly limited. In the embodiment illustrated in FIG. 2, the wall thickness tapers distally to aid in flexibility and trackability. In one embodiment, the second elongate member 21 may be a hypotube ground distally. As illustrated in FIG. 2, the core shaft 20 may be thicker at the proximal end 22 of the second elongate member 21, for example, to facilitate manipulation of the shaft assembly.

The composition of the second elongate member 21 is not particularly limited. The second elongate member 21 may be made from a metal, polymer or composite material, preferably a biocompatible material. The second elongate member 21 may comprise a shape memory metal or alloy such as a NiTi alloy (e.g., Nitinol).

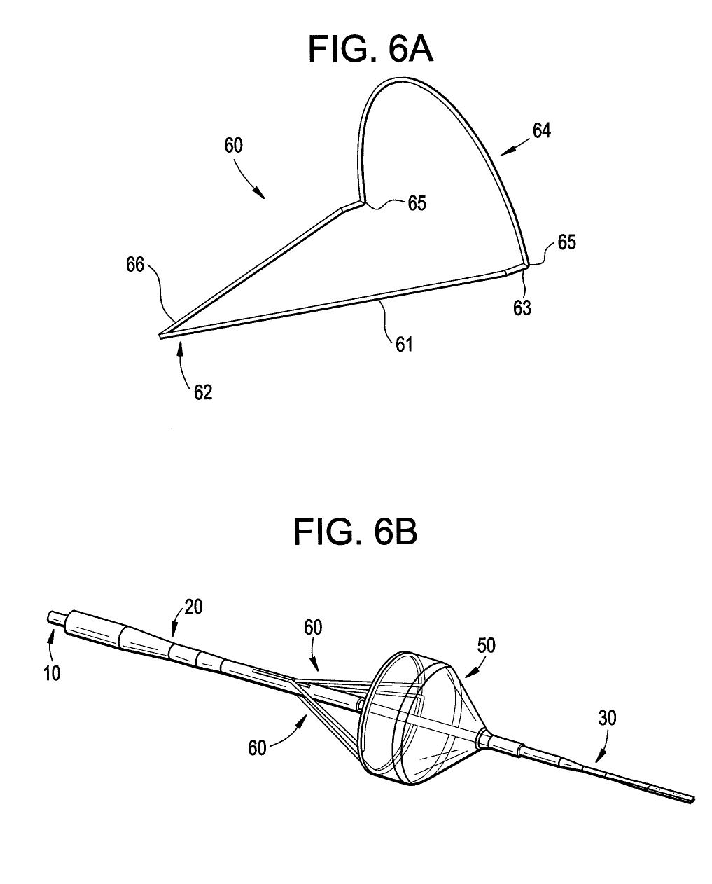

The core shaft 20 may be composed of multiple components. In addition to the lumen 26 that extends from the proximal end 22 to the distal end 23 of the second elongate member 21, the second elongate member 22 may also include a slot 24 that is adapted to align with and receive the tether attachment feature 14. When both of these features are present, and the actuator 10 is slidably received within the core shaft 20, the actuator 10 can translate proximally and distally but cannot rotate relative to the core shaft. This feature is illustrated in FIG. 4B and is discussed further below with regards to operation of the embolic protection device. For example, the slot 24 may accommodate at least a portion of the filter 50 support elements discussed above with respect to FIG. 8. The exact shape of the slot 24 is not particularly limited and may be formed by any means, such as a laser cut.

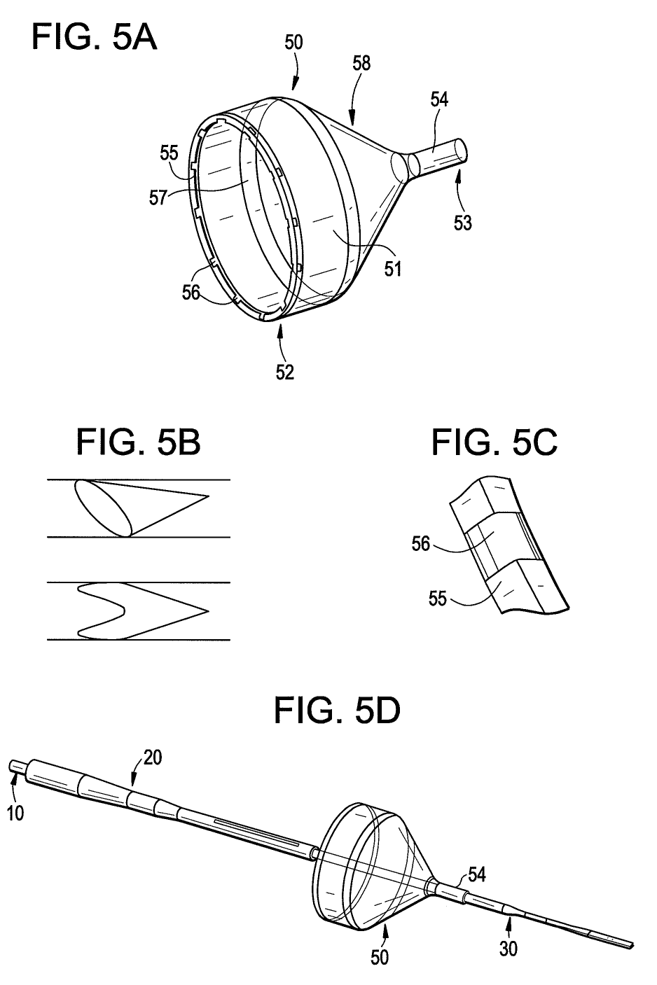

Referring now to FIG. 3, there is shown an exemplary embodiment of a guiding tip 30 in accordance with the present invention. The guiding tip 30 includes a tip body 31, a proximal end 32 and a distal end 33. The composition of the tip body 31 is not particularly limited. Typically, the entire tip is flexible relative to the first and second elongate member. In the embodiment illustrated in FIG. 3, the distal end 23 of the tip 30 is more flexible than the proximal end 32 as depicted by the varying cross sections. The composition of the tip body 31 may be any suitable material, as a metal, polymer or composite material, preferably a biocompatible material.

As shown, the guiding tip 30 has soft tip portion 34 for acute vessel access. The soft tip portion 34 may be provided to minimize discomfort for the patient when the embolic protection device is inserted into the patient. The composition of the soft tip portion 34 is not particularly limited. Examples of materials for the soft tip portion 34 include, but are not limited to, polymeric materials, such as PEBAX (polyether block polyamide), nylon, polyester, polyurethane, polyethylene or appropriate copolymers thereof. The soft tip portion 34 may be made of a radiopaque or non-radiopaque material.

Referring now to FIG. 4A, there is shown an exemplary embodiment of a shaft assembly 40 in accordance with the present invention. As described above, the shaft assembly 40 is a combination of the actuator 10, core shaft 20 and guiding tip 30. As illustrated in FIGS. 4A and B, the shaft assembly 40 is formed when the first elongate member 11 is slidably disposed within the lumen 26 of the of the second elongate member 21, and then the guiding tip 30 is coupled to the distal end 23 of the second elongate member 21. The guiding tip 30 may be coupled to the core shaft 20 by any means, such as a fixed bond or laser weld. In the illustrated embodiment, the coupled portion of the proximal end 32 of the guiding tip 30 is slidably disposed within the lumen 26 of the distal end 23 of the second elongate member 21 to decrease the profile of the shaft assembly 40. In FIG. 4, because the guiding tip 30 is coupled to the distal end 23 of the core shaft 20, within the lumen 26 of the core shaft 20, the guiding tip 30 would limit distal translation of the actuator 10 which is slidably disposed within the lumen of the core shaft 20.

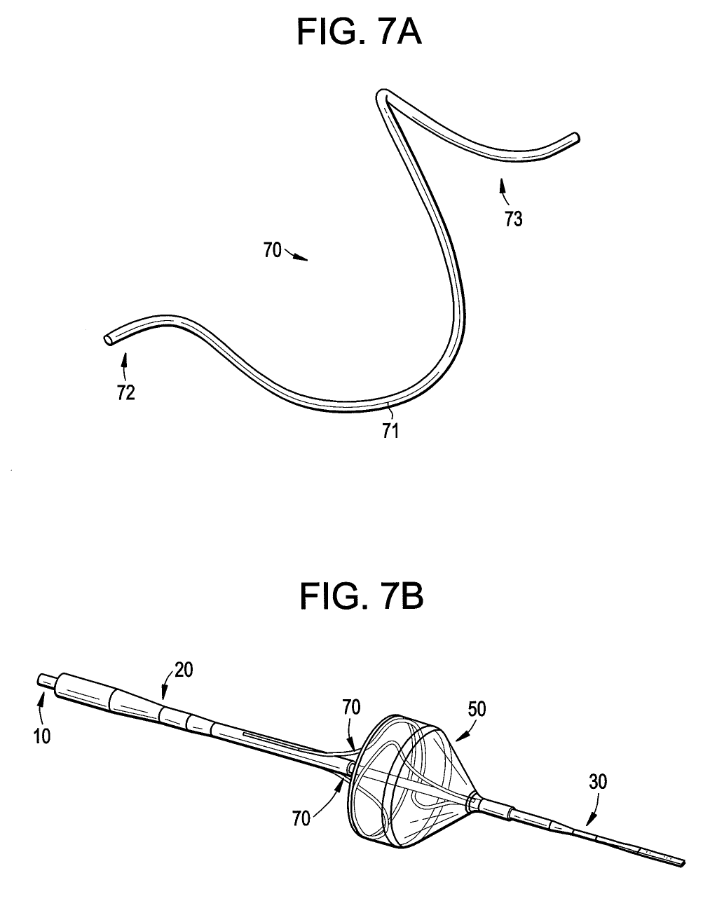

Referring now to FIG. 5A, there is shown an exemplary embodiment of an expandable filter 50 in accordance with the present invention. The filter 50 includes a filter membrane 51 which forms the body of the filter 50. The filter membrane 51 includes a proximal end 52 and a distal end 53. The filter 50 is movable between a low-profile, collapsed position and an expanded position, the expanded position illustrated, for example, in FIG. 5(d). In one embodiment of the invention, the filter 50 is sized for complete coverage of a vessel cross-section that allows passage of blood and blood components.

The composition of the filter member 51 is not particularly limited. For example, the filter membrane 51 may be formed from a compliant material, such as a flexible and/or elastic polymeric material, that has the ability to be stretched onto one or more support elements. Examples of the polymeric material utilized to create the filter member 51 include, but is not limited to, PEBAX (polyether block polyamide), nylon, polyester, polyurethane, polyethylene, ePTFE or appropriate copolymers thereof. The filter membrane 51 may also have shape memory properties to aid in deployment. For example, the filter membrane 51 may be made from a self-expanding or self-collapsing material, such as a shape memory polymer.

The filter 50 may also be partially of laminate construction comprising the membrane 51 coated with a coating (not shown) which is biocompatible. An example of such a filter is described in U.S. Patent Application Publication No. 2008/0167677, incorporated herein by reference. The coating may be of hydrophilic material. The filter 50 may have regions of varying hardness or stiffness.

The shape of the filter 50 in its expanded position is not particularly limited. In the illustrated embodiment, the filter 50 is shown in a funnel-like shape (that is, the distal end 53 has a conical shape with a wider cylindrical shape at the proximal end 52). In the embodiments illustrated in FIG. 5B, the filter 50a has an offset shape and the filter 50b has a wedge shape at the proximal end 52 to reduce bunching of the filter membrane 51, for example, when the filter 50 is in its low-profile collapsed position. The filter material bunch up in the collapsed position would be spread in the longitudinal direction rather than the radial.

As shown in FIG. 5A, when the filter 50 is in its expanded position, the filter membrane 51 forms a proximal inlet 57. The filter 50 expands for extension across a blood vessel such that blood passing through the blood vessel is delivered through the proximal inlet 57. In a preferred embodiment, the filter 50 has distal outlet pores 57 located near the distal end 53 of the filter membrane 51. Thus, the proximal inlet 57 allows blood and embolic material to enter the filter membrane 51, however, the distal outlet pores 58 allow through passage of blood but retain undesired embolic material within the filter membrane 51. The distal outlet pores 58 may be formed by any means, including laser cut. In an alternative embodiment, the filter membrane 51 is substantially composed of a porous material or a mesh material that provides relatively the same filtering capability as the distal outlet pores 57.

In an embodiment of the invention, the distal outlet pores 58 are sized to capture embolic material of a size large enough to impair the function of the organ receiving the blood downstream of the filter 50. Preferably, the distal outlet pores 58 are sized to capture embolic material of a size greater than 100 microns. More preferably, the distal outlet pores 58 are sized to capture embolic material of a size greater than 200 microns. Most preferably, the distal outlet pores 58 are sized to capture embolic material of a size greater than 500 microns.

As illustrated in FIGS. 5A and B, the proximal end 52 of the filter membrane 51 includes a membrane lip 55 and membrane slots 56 for securing the filter 50 to the shaft assembly 40 in its low-profile collapsed position. The membrane lip 55 may be inverted and heat welded in place. The membrane lip 55 may be asymmetric or bisymmetric to reduce material stack up. In one embodiment, the membrane lip 55 and membrane slots 56 are adapted to receive a tether 60 for securing the filter 50, in tension, on the shaft assembly 40 when the filter 50 is in its low-profile collapsed position, as discussed below.

In the illustrated embodiment, the filter 50 includes a distal cone 54. As shown in FIG. 5(d), the distal cone 54 is disposed on the guiding tip 30 to attach the filter 50 to the shaft assembly 40. The method of attaching the distal cone 54 to the guiding tip 30 is not particularly limited. The distal cone 54 may be fixedly attached guiding tip 30 such that the filter 50 cannot translate proximally or distally relative to the guiding tip. The distal cone 54 may also be rotatably attached to the guiding tip 30, such that the filter 50 has freedom to rotate relative to the guiding tip 30.

The distal cone 54 may be composed of the same material as the filter membrane 51. In another embodiment, the distal cone 54 is composed of a different material than the filter membrane 51, such that the filter membrane 51 is gathered into or connected to the distal cone 54. For example, the distal cone 54 may comprise a sleeve that is disposed on guiding tip 30.

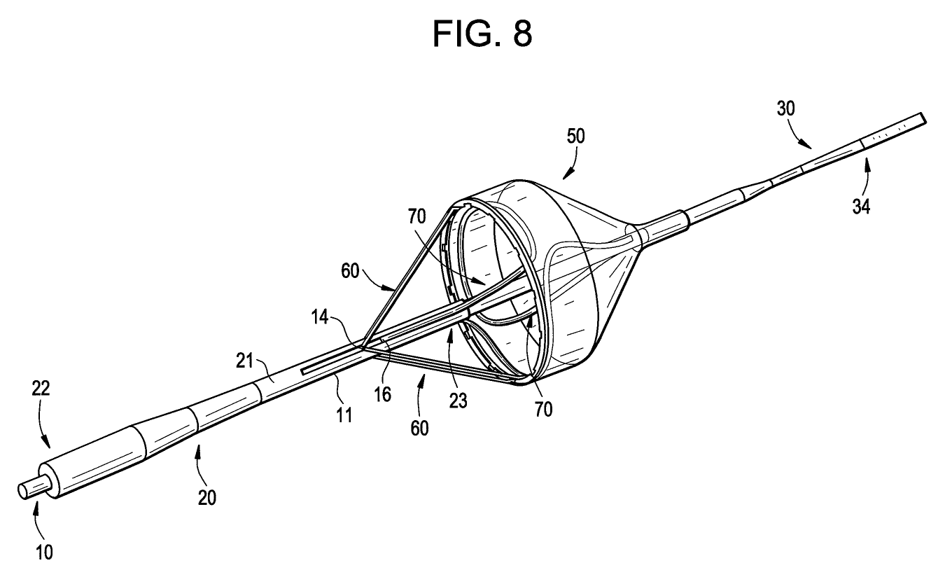

Referring now to FIG. 6A, there is shown an exemplary embodiment of a tether 60 in accordance with the present invention. The tether 60 includes a flexible tether body 61 having a proximal end 62 and a distal end 63. The composition of the tether body 61 is not particularly limited. Preferably, the tether body 61 comprises a metal, polymer or composite material. The tether body 61 may be made from a shape memory material. Examples of the shape memory material include a shape memory alloy, such as a nickel titanium alloy like Nitinol, or a shape memory polymer.

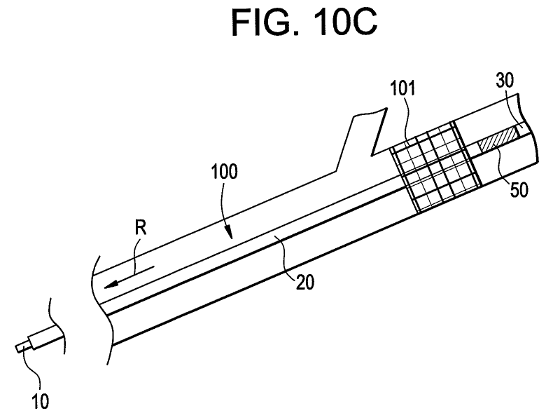

In the illustrated embodiment, the distal end 63 of the tether body 61 includes a loop section 64. As discussed above, the filter membrane may include a membrane lip 55 and membrane slots 56 which are adapted to receive the tether 60, particularly the loop section 64. As illustrated in FIG. 9, the crimped portions 65 at the end of each loop section 64 line up with the membrane slots 56, such that the loop section 64 may be slidably received into membrane lip 55. In some embodiments, the middle of the loop portion 64 may also have a hinge, not shown, to facilitate collapsing the tether body 61.

The tether 60 may be used to hold the filter membrane 51, in tension, on the shaft assembly 40 when the filter 50 is in its low-profile collapsed position during delivery to a treatment site. For example, the tether body 61 may be self collapsing to hold the filter 50 in tension on the shaft assembly 40.

As illustrated in FIG. 6B, more than one tether 60 may be employed. For example, in one embodiment, a first and second tether body 61 are used such that substantially all of the circumference of the filter membrane 51 formed by the membrane lip 55 is supported by the loop portions of the first and second tether bodies.

The tether 60 also includes means for attaching the filter to the shaft assembly 40. In FIG. 6A, the tether body 61 includes an attachment point 66. The tether attachment point 66 corresponds to the tether attachment feature 14 on the first elongate member 11 of the actuator 10 and is not particularly limited. The means for attaching the tether 60 to the actuator 10 is not particularly limited. Examples include, but are not limited to, a heat weld, a laser weld, a fixed bond, or a friction fit.

As shown in the embodiment illustrated in FIGS. 8 and 9, the tether 60 is coupled to both the actuator 10 and the filter 50. Because the filter 50 is attached to the guiding tip 30 and the guiding tip is attached to the core shaft 20, proximal translation of the actuator 10 relative to the core shaft 20 places a proximal force on the tether 60 to collapse the tether 60 toward the shaft assembly and into a low-profile, collapsed position. The method of collapsing the tether 60 is not particularly limited and is based on the shape of the tether. In the example illustrated in FIG. 11, when the tether 60 is pulled in the proximal direction indicated by arrow 69, the loop portion 64 “folds” in the distal direction indicated by arrow 68 towards the shaft assembly and may be “pinched” closed.

For the same reason, distal translation of the actuator 10 relative to the core shaft 20 expands the tether 60 away from the shaft assembly and into an expanded position. Accordingly, the tether 60 may be used to close the proximal inlet 57 of the filter 50.

In one embodiment, at least a portion of the tether body 61 is received within the slot 24 of the second elongate member 21 when the filter 50 is in its low-profile, collapsed position. In a preferred embodiment, when the filter 50 is in its low-profile, collapsed position, the tether body 61 is substantially received within the slot 24 of the second elongate member 21 of the core shaft 20. Preferably, the portion of the tether body 61 that is collapsed along the distal end 73 of the second elongate member 72 is completely received within the slot 24.

Referring now to FIG. 9, the guiding tip 30 may also function as an abutment/placement feature for translation of the tether 60 when the guiding tip 30 is coupled to the distal end of the core shaft 20 within the lumen of the second elongate member 21. In other words, distal translation of actuator 10 within the core shaft 20 will be limited by the guiding tip 30, and when the tether 60 is coupled to actuator 10, a portion of the tether 60 may abut the guiding tip 30.

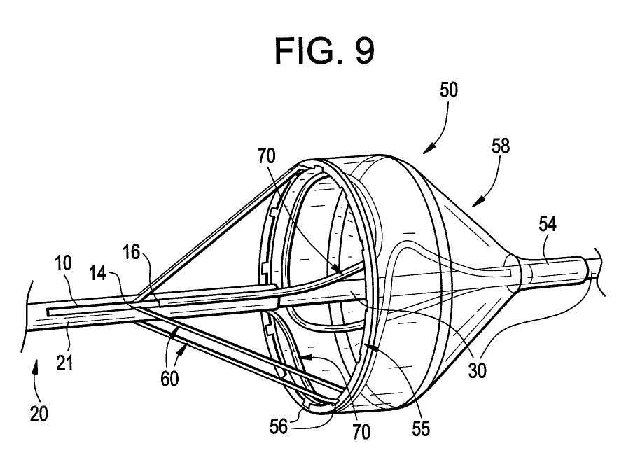

Referring now to FIG. 7A, there is shown an exemplary embodiment of a frame 70 in accordance with the present invention. The frame 70 includes a flexible frame body 71 having a proximal end 72 and a distal end 73. The composition of the frame body 71 is not particularly limited. Preferably, the frame body 71 comprises a metal, polymer or composite material. The frame body 71 may be made from a shape memory material. Examples of the shape memory material include a shape memory alloy, such as a nickel titanium alloy like Nitinol, or a shape memory polymer.

In one embodiment, the frame 70 is used to support the filter membrane 51 when the filter 50 is in its expanded position as shown, for example, in FIG. 7B. The form of the frame body 71 is not particularly limited. As shown, the frame body 71 can be a spiral.

The frame 70 may also be used to aid in expanding the filter 50. For example, the frame body 71 may be made from a self-expanding material, such as one of the shape memory materials discussed above.

As illustrated in FIG. 7B, for example, more than one frame 70 may be employed. For example, it should be understood that using multiple spiral frame bodies 71 would essentially form a complete circular (or elliptical) support. However, an increased number of spirals may also reduce “parking” space of the filter 50 in its collapsed position and/or increase the profile of the filter 50 in its collapsed position. In a preferred embodiment, a first and second frame body 71 are used.

Referring now to FIG. 9 and to FIG. 1, the proximal end 72 of the frame body 71 is coupled to the frame attachment feature 16 that is located on the distal end 13 of the first elongate member 11. The method of attaching the proximal end 72 of the frame body 71 to the frame attachment feature 16 is not particularly limited. In addition, the distal end 73 of the frame body 71 is coupled to the guiding tip 30. The method of attaching the distal end 73 of the frame body 71 to the guiding tip 30 is not particularly limited.

In the embodiment illustrated in FIG. 9, the frame 70 is coupled to both the actuator 10 and guiding tip 30. Because the guiding tip 30 is attached to the core shaft 20, proximal translation of the actuator 10 relative to the core shaft 20 collapses the frame 70 towards the shaft assembly and into a low-profile, collapsed position, and distal translation of the actuator 10 relative to the core shaft 20 expands the frame 70 away from the shaft assembly and into an expanded position.

In one embodiment, when the frame 70 is in its collapsed position, at least a portion of the frame is received within the second elongate member 21 by way of the slot 24. In a preferred embodiment, when the frame 70 is in its collapsed position, the portion of the tether body 71 that is collapsed along the length of the distal end 23 of the core shaft 20 is received within the slot 24 of the second elongate member 21.

Referring to FIG. 9, the guiding tip 30 may also function as an abutment/placement feature for translation of the frame 70 when the guiding tip 30 is coupled to the distal end of the core shaft 20 within the lumen of the second elongate member 21. In other words, distal translation of actuator 10 within the core shaft 20 will be limited by the guiding tip 30, and when the frame 70 is coupled to actuator 10, a portion of the frame 70 may abut the guiding tip 30.

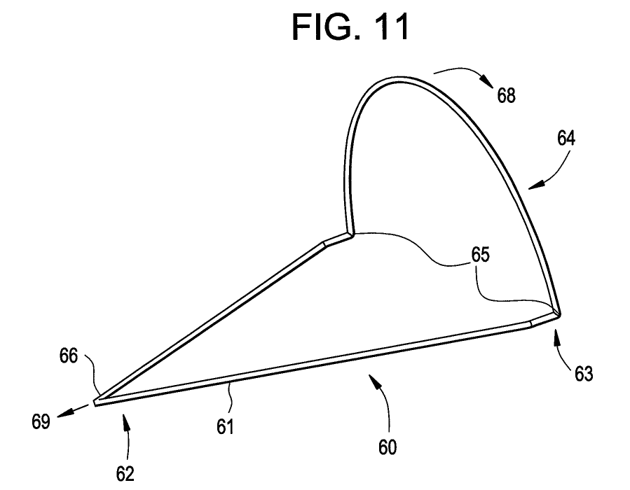

Referring now to FIGS. 10A-C, an illustrated example of the embolic protection device of the present invention in use is described. In FIGS. 10A-C there is shown a cross-sectional view of a vessel V, wherein the vessel includes a stenosed region S.

Referring generally to FIGS. 10A-C, an embolic protection device 100 is shown having an actuator 10, a core shaft 20, a guiding tip 30 and a filter 50 such as those illustrated in FIGS. 1, 2, 3 and 5, respectively. The direction of the flow of blood within the vessel V is marked by the arrow B.

Prior to entering the vasculature of the patient and delivering the filter 50 to the stenosed region S shown in FIG. 10A, the filter 50 must be prepared for delivery. Preparing the filter for delivery includes collapsing the filter 50 to its low-profile, collapsed position. The filter 50 may be collapsed by pulling the actuator 10 in a proximal direction relative to the core shaft 20 while maintaining the position of the core shaft 20. The filter 50 may be collapsed under a saline bath.

The means for maintaining the filter 50 in the low-profile, collapsed position are not particularly limited. In one embodiment, the filter 50 is held in the collapsed position for delivery using a clip device, not shown. The clip device would be coupled between the actuator 10 and the core shaft 20 at the proximal end 22 of the embolic protection device 100 to hold in place the positions of the actuator 10 and core shaft 20 relative to one another.

Referring now to FIG. 10A, an embolic protection device 100 is shown having a proximal end 22 outside of a patient's body and a distal end 23 within the vasculature of a patient's body. The distal end 23 is located at a position proximal to the stenosed region S of the vessel V. The filter 50 is shown in its low-profile, collapsed condition for delivery in the direction D towards the distal deployment position P. A tether, not shown, may be employed to hold the filter 50, in tension, against the shaft assembly during delivery. Holding the filter 50 in tension reduces the profile of the embolic protection device 100.

The operator may advance the embolic protection device 100 through the vasculature to the distal deployment position P by distally translating the core shaft 20. The guiding tip 30, which is attached to the distal end of the core shaft, preferably has a soft tip portion, not shown, for acute vessel access and to provide comfort to the patient.

Referring now to FIG. 10B, the embolic protection device 100 has crossed the stenosed region S and the filter 50 has been expanded at the distal deployment position P. Filter 50 is expanded by distally translating the actuator 10 relative to the core shaft 20. For example, if a clip was used to hold in place the positions of the actuator 10 and the core shaft 20 relative to one another, the clip may be removed by the operator. Then, the actuator 10 may be translated distally while holding the core shaft 20 in place.

If a self-expanding frame, not shown, is coupled between the actuator 10 and the guiding tip 30, the self-expanding frame will aid in expanding the filter 50 and provide a support for the body of the filter in its expanded position. Conveniently, when at least a portion of the frame is accommodated within core shaft 20, as described above, the embolic protection device 100 has a reduced profile to cross a lesion within a vessel, such as the stenosis S.

Once the filter 50 has been expanded, the filter 50 is in a position to filter emboli from blood flowing through the vessel V in the direction B, and an interventional procedure may be performed at or near the stenosed region S. In this respect, the core shaft 20 is capable of being used similar to a guidewire in order to translate a medical device along the length of the core shaft 20 to the stenosed region. The interventional procedure that may be performed and the medical device that device that may be used is not particularly limited. Examples include known devices such as balloon catheter and stent. In the illustrated embodiment, a stent 101 is translated to the stenosed region S for expansion by a known method. During the procedure, the expanded filter 50 filters the blood flowing through the vessel to capture emboli that may be dislodged.

Referring now to FIG. 10C, after the interventional procedure is complete, such as expanding a stent 101 at the stenosed region S, the filter 50 is collapsed and the embolic protection device 100 is withdrawn from the patient in the retrieval direction R. To collapse the filter 50, the actuator 10 is pulled proximally relative to the core shaft 20. When the filter 50 is collapsed, captured material that was too large to flow through the filter 50 is sealed within the body of the filter 50. The filter 50 is then held in the low-profile, collapsed position by maintaining the positions of the actuator 10 and the core shaft 20 relative to one another. The same clip device, not shown, could be used for this purpose. Once the filter 50 is collapsed, the embolic protection device 100 may be withdrawn from the patient.

In accordance with the above description, an embolic protection device having a filter that is capable of being delivered and retrieved from a treatment site in a low-profile, collapsed condition without the use of a delivery catheter or a retrieval catheter has been realized.

The invention is susceptible to various modifications and alternative means, and specific examples thereof have been shown by way of example in the drawings and are herein described in detail. It should be understood, however, that the invention is not to be limited to the particular devices or methods disclosed, but to the contrary, the invention is to cover all modifications, equivalents, and alternatives falling within the spirit and scope of the claims.

What is claimed is:

1. An embolic protection device comprising:
a first elongate member having a proximal end and a distal end;
a second elongate member having a lumen, a proximal end a distal end, the first elongate member slidably disposed within the lumen of the second elongate member;
a guiding tip having a proximal end and a distal end, the proximal end of the guiding tip coupled to the distal end of the second elongate member; and
an expandable filter disposed on the guiding tip;
wherein the filter is movable between a low-profile, collapsed position and an expanded position by distally translating the first elongate member relative to the second elongate member.

2. The embolic protection device according to claim 1, wherein the first elongate member can translate distally and proximally relative to the second elongate member but cannot rotate relative to the second elongate member.

3. The embolic protection device according to claim 1, wherein the guiding tip limits the distal translation of the first elongate member within the lumen of the second elongate member.

4. The embolic protection device according to claim 1, further comprising a frame having a proximal end and a distal end, wherein the proximal end of the frame is coupled to the first elongate member and the distal end of the frame is coupled to the guiding tip.

5. The embolic protection device according to claim 4, wherein the frame is fixedly attached to the guiding tip.

6. The embolic protection device according to claim 4, wherein the frame is self-expanding.

7. The embolic protection device according to claim 4, wherein the frame is movable between a low-profile, collapsed position and an expanded position.

8. The embolic protection device according to claim 7, wherein the filter is supported by the frame when the frame is in its expanded position.

9. The embolic protection device according to claim 7, wherein at least a portion of the frame is received within the lumen of the second elongate member when the frame is in its low-profile, collapsed position.

10. The embolic protection device according to claim 1, further comprising a tether having a proximal end and a distal end, wherein the proximal end of the tether is coupled to the first elongate member and the distal end of the tether is coupled to the filter.

11. The embolic protection device according to claim 10, wherein the tether is self-collapsing.

12. The embolic protection device according to claim 10, wherein the tether is movable between a low-profile, collapsed position and an expanded position.

13. The embolic protection device according to claim 10, wherein the tether holds the filter, in tension, on the embolic protection device when the filter is in its low-profile, collapsed position.

14. The embolic protection device according to claim 12, wherein at least a portion of the tether is received within the lumen of the second elongate member when the tether is in its low-profile, collapsed position.

15. The embolic protection device according to claim 1, wherein the filter comprises a flexible filter membrane having a proximal end and a distal end.

16. The embolic protection device according to claim 15, wherein a distal end of the filter membrane comprises pores for filtration.

17. The embolic protection device according to claim 15, wherein the filter membrane is self-expanding.

18. The embolic protection device according to claim 1, wherein the guiding tip has a proximal end and a distal end, the distal end of the guiding tip comprising a soft portion that is relatively more flexible.

19. A method for filtering emboli from blood flowing at a delivery location within a vasculature comprising:
providing the embolic protection device according to claim 1;
collapsing the filter by translating the first elongate member proximally relative to the second elongate member to move the filter into the low-profile, collapsed position;
advancing the embolic protection device to the delivery location within the vasculature;
deploying the filter by translating the first elongate member distally relative to the second elongate member to move the filter to the expanded position;
performing an interventional procedure at a position proximal to the delivery location;
collapsing the filter by translating the first elongate member proximally relative to the second elongate member to move the filter to the low-profile, collapsed position after the interventional procedure; and
withdrawing the embolic protection device from the vasculature.

20. The method for filtering emboli from blood flowing through a vasculature according to claim 19, wherein the interventional procedure comprises advancing a stent over the second elongate member to a position proximal to the delivery location.
